(19) United States
(12) Patent Application Publication
(10) Pub. No.: US 2024/0094519 A1
(43) Pub. Date: Mar. 21, 2024

(54) ACETALS OF TRICYCLODECANE ALCOHOLS AS IMMERSION LIQUID MEDIA

(71) Applicant: Carl Zeiss Jena GmbH, Jena (DE)

(72) Inventors: Matthias KRIEG, Jena (DE); Jakob Benedikt Jäger, Jena (DE)

(73) Assignee: Carl Zeiss Jena GmbH, Jena (DE)

(21) Appl. No.: 18/457,190

(22) Filed: Aug. 28, 2023

(30) Foreign Application Priority Data
Aug. 29, 2022 (DE) 10 2022 121 826.0

(51) Int. Cl. G02B 21/33

(52) U.S. Cl. CPC G02B 21/33

(57) ABSTRACT

An immersion liquid, especially for microscopy, contains one or more acetals of tricyclodecane alcohols as its main constituent, wherein a tricyclodecane alcohol is a compound of which the structure has the base skeleton of tricyclo[5.2.1.0^{2,6}]decane and at least one OH group, and wherein all OH groups of the tricyclodecane alcohol have been acetalized in the acetal. Also described are 3(4),8(9)-bis(tetrahydropyran-2-yloxymethyl)tricyclo[5.2.1.0^{2,6}]decane or 8-tetrahydropyran-2-yloxymethyltricyclo[5.2.1.0^{2,6}]decane and the preparation thereof, and the use of acetals of tricyclodecane alcohols as the main constituent of an immersion liquid. A process for preparing an acetal of a tricyclodecane alcohol involves reacting a tricyclodecane alcohol with an acetalizing agent in the presence of an acid.

### CROSS-REFERENCE TO RELATED APPLICATIONS

This application claims priority to German Application No. 102022121826.0, filed on Aug. 29, 2022, the content of which is hereby incorporated by reference in its entirety.

### BACKGROUND OF THE INVENTION

### Technical Field

The invention relates to immersion liquids, especially for light microscopy, and to processes for preparing components suitable therefor.

### Description of Related Art

Immersion oils are an important component, especially in modern light microscopy systems. Such immersion oils must meet particular demands, especially with regard to: refractive index, dispersion, optical transparency (up to 365 nm), residual fluorescence, viscosity, removability from optical instruments, nontoxicity, environmental compatibility etc. These demands are met by synthetic compounds containing specific aliphatic ring systems.

DE 197 05 978 A1 describes an immersion oil for microscopy. It contains esters or ethers with tricyclodecane structure as main constituents and one or more high-boiling liquids as secondary constituents. In particular, the following are described: esters and ethers that derive from tricyclodecanemethanol (TCD-M) and tricyclodecanedimethanol (TCD-DM) and are processed to give immersion oils. The immersion oils are halogen-free, have high UV transparency and, because of the vacuum distillability of the components used, feature low intrinsic fluorescence.

EP 0 209 621 A2 specifies examples both of halogen-containing and halogen-free immersion oils. Two examples of halogen-free immersion oils contain tricyclodecanol as a secondary component. However, no figures in the document are given with regard to UV transparency (at wavelengths below 400 nm). Moreover, kinematic viscosity with values of 1000 and 2000 mm²/s is too high for most applications, meaning that trapped air bubbles can very easily occur on use.

### SUMMARY OF THE INVENTION

### Object of the Invention

The preparation of the tricyclodecanemethanol esters described in DE 197 05 978 A1 is complex; in particular, it entails elevated reaction temperatures and prolonged reaction times, and complex purification of the primary reaction products.

There is therefore a need to provide comparable compounds for use as main constituents of immersion liquids that can be produced in a simpler manner.

The resultant immersion liquids are to have, among other properties, Abbe numbers between 40 and 50 with simultaneously low intrinsic fluorescence and high UVA transparency.

This object is achieved by acetals of tricyclodecane alcohols and the use thereof as main constituents of immersion liquids, especially for microscopy, but also for other optical applications, for example immersion lithography.

The invention accordingly provides the immersion liquid as described below. Preferred embodiments thereof are also defined below.

Moreover, the invention provides the acetals mentioned as novel compounds, the use thereof as the main constituent of immersion liquids, and a process for preparing the acetals.

### Advantageous Effects of the Invention

The acetals of tricyclodecane alcohols that are present as the main constituent in the immersion liquid according to the invention can be prepared in a particularly simple manner.

The compounds are prepared by acetalization of tricyclodecane alcohols in the presence of an acid as catalyst, and in particular requires only low reaction temperatures, and features a high reaction rate, such that the required reaction times are short.

A particularly favourable reaction is that of tricyclodecanedimethanol with 3,4-dihydro-2H-pyran (DHP) to form the corresponding bisacetal.

The reaction temperature is very favourable here for conversion and batch time (the reaction can be conducted at room temperature, i.e. 20 to 25° C.), with simultaneously short reaction time and high selectivity and yield.

Lowering the reaction temperature has multiple benefits: There is no need for a reactor heating time, the energy demand in the preparation is lower, there is no need to work with solvents and simultaneously hot surfaces (explosion range/ignition temperatures), and hence safety-related construction measures may be less significant. Moreover, lower reaction temperatures increase selectivities since less energy is available for unwanted side reactions to be able to proceed. (The reaction barriers of the desired target reaction are generally lower than those of the side reactions; this effect can be maximized by even lower reaction temperatures.)

Moreover, the reactions can be conducted using solvents that can be disposed of without difficulty on an industrial scale (because they are inexpensive, nonhazardous and easy to recycle or dispose of).

In the preparation of the acetals that are present as the main constituent in the immersion liquid according to the invention, there is largely a free choice of solvent since no particular boiling temperature has to be attained during the reaction, i.e. it is possible to use solvents that enable the greatest possible solubility of the reactants. It is thus possible to use solvents that represent a minimum potential endangerment to workers, have good distillability (and hence removability from the reaction mixture) and recyclability, and can be disposed of inexpensively.

In the reaction of a tricyclodecane alcohol with 3,4-dihydro-2H-pyran or other acetalizing agents, it is possible to work with a slight to large molar excess (10-100%) of dihydropyran in order to allow the reaction to proceed as completely as possible. It is possible here to remove both the solvent (for instance ethyl acetate) and the excess dihydropyran in one step under the same conditions since the boiling points of the dihydropyran and of the solvent can be very dose to one another. By comparison with the tricyclodecanemethanol esters and ethers described in DE 197 05 978 A1, it is therefore possible to dispense with one step (the separate removal of excess alcohol) and to gain about 1 week of production time.

The immersion liquids according to the invention are thus notable for ease of preparability of the main constituents. At the same time, they meet the requirements according to standard ISO 8036:2015-06.

The invention also includes the following embodiments:
1. Immersion liquid, especially for microscopy, comprising one or more acetals of tricyclodecane alcohols as its main constituent, wherein a tricyclodecane alcohol is a compound of which the structure has the base skeleton of tricyclo[5.2.1.0^{2,6}]decane and at least one OH group, and wherein all OH groups of the tricyclodecane alcohol have been acetalized in the acetal.
2. Immersion liquid according to embodiment 1, comprising one or more acetals of 8-hydroxymethyltricyclo[5.2.1.0^{2,6}]decane or 3(4),8(9)-bis(hydroxymethyl)-tricyclo[5.2.1.0^{2,6}]decane as its main constituent.
3. Immersion liquid according to embodiment 2, comprising 8-tetrahydropyran-2-yloxymethyltricyclo[5.2.1.0^{2,6}]decane and/or 3(4),8(9)-bis(tetrahydropyran-2-yloxymethyl)tricyclo[5.2.1.0^{2,6}]decane as its main constituent.
4. Immersion liquid according to any of embodiments 1 to 3, comprising one or more high-boiling liquid(s) as secondary constituents.
5. Immersion liquid according to embodiment 4, wherein the high-boiling liquids are selected from paraffin oil, plasticizers, polypropylene glycols, dioctyl sebacate, diisopropylnaphthalene, di(propylene glycol-1,2) benzoate and mixtures thereof.
6. Immersion liquid according to any of embodiments 1 to 5, wherein the proportion of the one or more acetals of tricyclodecane alcohols is more than 50 per cent by weight, preferably at least 60 per cent by weight.
7. 3(4),8(9)-Bis(tetrahydropyran-2-yloxymethyl)tricyclo[5.2.1.0^{2,6}]decane or 8-tetrahydropyran-2-yloxymethyltricyclo[5.2.1.0^{2,6}]decane.
8. Use of acetals of tricyclodecane alcohols as the main constituent of an immersion liquid.
9. Process for preparing acetals of tricyclodecane alcohols, comprising the reaction of a tricyclodecane alcohol with an acetalizing agent in the presence of an acid.
10. Process according to embodiment 9, wherein the reaction is effected at a temperature within a range from 20 to 30° C.
11. Process according to embodiment 9 or 10, wherein the acetalizing agent is 3,4-dihydro-2H-pyran.

### DETAILED DESCRIPTION OF THE INVENTION

### Tricyclodecane Alcohols and Acetals Thereof

The immersion liquid according to the invention contains acetals of tricyclodecane alcohols as its main constituent. These acetals are also referred to hereinafter by way of simplification as “tricyclodecane acetals” (TCD acetals).

Tricyclodecane alcohols (which are also referred to hereinafter by way of simplification as “TCD alcohols”) are understood to mean compounds of which the structure has the base skeleton of tricyclo[5.2.1.0^{2,6}]decane and at least one OH group. The structure of the tricyclodecane alcohol preferably has one, two or three OH groups.

Examples of suitable TCD alcohols are the compounds 1 to 14 shown below, the names of which are given in the table below. This table also indicates the identifiers by which the compounds are in some cases referred to as commercially available products (i.e., for example, compound 1 is also referred to as “TCD alcohol A”).

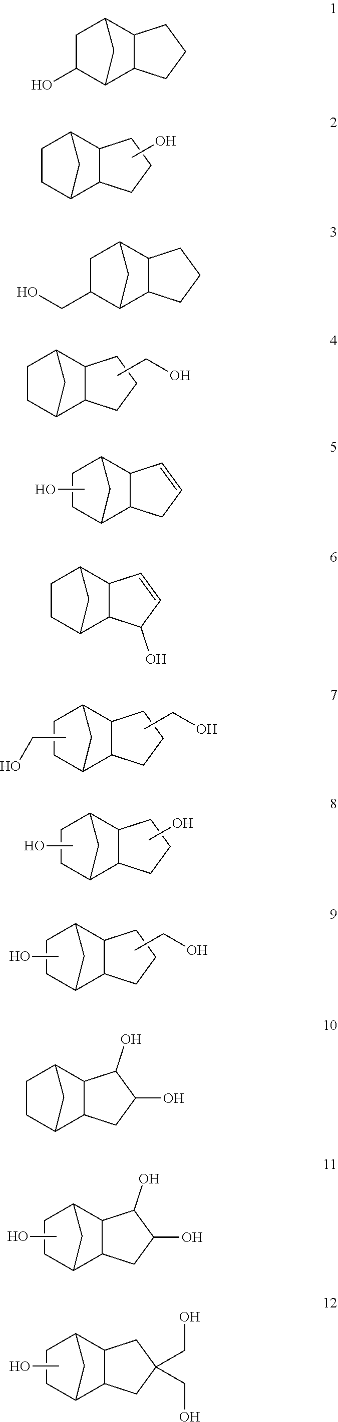

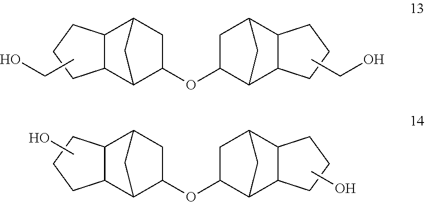

| No. | Name | Identifier |
|---|---|---|
| 1 | 8-Hydroxytricyclo[5.2.1.0^{2,6}]decane | A |
| 2 | 3(4)-Hydroxytricyclo[5.2.1.0^{2,6}]decane | |
| 3 | 8-Hydroxymethyltricyclo[5.2.1.0^{2,6}]decane | M |
| 4 | 3(4)-Hydroxymethyltricyclo[5.2.1.0^{2,6}]decane | |
| 5 | 8(9)-Hydroxytricyclo[5.2.1.0^{2,6}]dec-3-ene | E |
| 6 | 5-Hydroxytricyclo[5.2.1.0^{2,6}]dec-3-ene | |
| 7 | 3(4),8(9)-Bis(hydroxymethyl)tricyclo[5.2.1.0^{2,6}]decane | DM |
| 8 | 3(4),8(9)-Dihydroxytricyclo[5.2.1.0^{2,6}]decane | |
| 9 | 8(9)-Hydroxy-4(5)-hydroxymethyltricyclo[5.2.1.0^{2,6}]decane | OM |
| 10 | 3,4-Dihydroxytricyclo[5.2.1.0^{2,6}]decane | |
| 11 | 8(9),3,4-Trihydroxytricyclo[5.2.1.0^{2,6}]decane | TO |
| 12 | 8(9)-Hydroxy-4,4-bis(hydroxymethyl)tricyclo[5.2.1.0^{2,6}]decane | |
| 13 | 3(4),3′(4′)-Bis(hydroxymethyl)di(tricyclo[5.2.1.0^{2,6}]dec-8,8′-yl) ether | |
| 14 | 3(4),3′(4′)-Dihydroxydi(tricyclo[5.2.1.0^{2,6}]dec-8,8′-yl) ether | |

The acetals of the TCD alcohols 3 and 7 (“TCD alcohol M” and “TCD alcohol DM”) are preferred, especially the acetals formed with 3,4-dihydro-2H-pyran (including 8-tetrahydropyran-2-yloxymethyltricyclo[5.2.1.0^{2,6}]decane).

The bisacetals of the TCD alcohol 7 are particularly preferred, especially the bisacetal formed with 3,4-dihydro-2H-pyran (3(4),8(9)-bis(tetrahydropyran-2-yloxymethyl)tricyclo[5.2.1.0^{2,6}]decane).

In the preferred examples, the main constituents of the immersion liquid thus consist of acetals of 8-hydroxymethyltricyclo[5.2.1.0^{2,6}]decane (also referred to as tricyclodecanemethanol or “TCD alcohol M”) or of 3(4),8(9)-bis(hydroxymethyl)tricyclo[5.2.1.0^{2,6}]decane (also referred to as tricyclodecanedimethanol or “TCD alcohol DM”), since these compounds are vacuum-distillable because of their molecular structure and hence are preparable with high purity. It is thus possible to prepare immersion liquids with minimal intrinsic fluorescence from the acetals of TCD alcohol M and TCD alcohol DM.

For the preparation of the TCD acetals, it is possible to make use of the following TCD alcohols that are available on an industrial scale:
8(9)-hydroxymethyltricyclo[5.2.1.0^{2,6}]decane (TCD alcohol M) and

3(4),8(9)-bis(hydroxymethyl)tricyclo[5.2.1.0^{2,6}]decane (TCD alcohol DM), which are commercially available. These are each (regio- and stereo-)isomer mixtures that conform to the following structural formula:

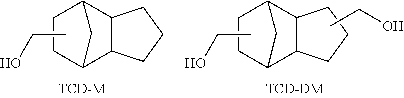

By acetalizing the TCD alcohols with acetalizing agents such as 3,4-dihydro-2H-pyran by customary methods, it is possible to synthesize the TCD acetals.

The acetalization is effected by mixing the TCD alcohol with the acetalizing agent, preferably 3,4-dihydro-2H-pyran, in a suitable solvent and adding an acid as catalyst.

Suitable solvents are in principle all solvents that dissolve the starting materials and do not disrupt the desired reaction. Examples of suitable solvents are ethyl acetate, dichloromethane, isoamyl acetate, and THF. Ethyl acetate is particularly preferred.

The reaction can be effected at room temperature (20 to 25° C.), i.e. without specific heating of the reaction mixture. The reaction time is preferably 30 minutes to 10 hours, more preferably 1 to 3 hours. If a rise in the reaction rate is desired, the reaction mixture can be heated to a temperature of 30 to 60° C., preferably 40 to 50° C. However, this is advantageously unnecessary.

The reaction preferably precedes at standard pressure, under air atmosphere; suitable reaction vessels may consist of glass or steel.

The proportion of the tricyclodecane acetals is at least 50 per cent by weight of the overall immersion liquid in examples in which the immersion liquid is a two-substance mixture, and at least 40 per cent by weight in the examples in which the immersion liquid is a three-substance or multisubstance mixture. The immersion liquid may also contain two or more tricyclodecane acetals, in which case the sum total of all tricyclodecane acetals is at least 40 per cent by weight of the immersion liquid.

Because of the ring structure of tricyclodecane (TCD), the tricyclodecane acetals have a relatively high refractive index with a simultaneously high Abbe number and are thus of excellent suitability as main component for immersion liquids. An immersion liquid containing TCD acetals as main constituents can therefore be halogen-free.

A particularly suitable main constituent for immersion liquids has been found to be the bisacetal of tricyclodecanedimethanol formed with 3,4-dihydro-2H-pyran (3(4),8(9)-bis(tetrahydropyran-2-yloxymethyl)tricyclo[5.2.1.0^{2,6}]decane); the proportion by weight of this bisacetal in the immersion liquid here is preferably at least 60 per cent by weight, more preferably at least 70 per cent by weight, especially preferably at least 80 per cent by weight.

### Acid

Suitable acid catalysts for the acetalization of the TCD alcohols are in principle all sufficiently strong acids, specifically both mineral acid (such as aqueous conc. hydrochloric acid, anhydrous conc. sulfuric acid) and organic acids of better solubility (for example sulfonic acids (para-toluenesulfonic acid, PTSA)). In particular, acids having a pKa of ≤1, preferably ≤0, are suitable. Weaker acids, for example acetic acid, show no effect as a catalyst addition.

### Immersion Liquids/Other Constituents

The immersion liquid according to the invention contains, as main constituents, one or more high-boiling liquid(s) as further component(s) for adjustment of the refractive index.

The high-boiling liquid may be selected from paraffin oil, plasticizers, polypropylene glycols, dioctyl sebacate, diisopropylnaphthalene, di(propylene glycol-1,2) benzoate and mixtures thereof.

In principle, the immersion liquid may be composed of two or more TCD acetals as main constituent and two or more high-boiling liquids for adjusting the refractive index as secondary constituent. Since, however, the starting substances have to be provided in high purity for low residual fluorescence and the amounts used are relatively small, particular preference is given to two-substance mixtures from the point of view of low production costs.

The proportion by weight of the TCD acetals in the immersion liquid in the case of two-component mixtures is preferably between 60 and 99 per cent by weight, and in the case of three-component mixtures is preferably between 50 and 70 per cent by weight.

The immersion liquids are produced by simply mixing the TCD acetal with suitable high-boiling liquids, for example plasticizers, paraffin oils, polypropylene glycols etc., to give the on-spec values for the refractive index.

The TCD acetals used typically have boiling points of 120° C. to 160° C. at <1·10⁻⁴ hPa.

The immersion liquid according to the invention typically has the following physical properties:
Refractive index: 1.5165-1.5195, preferably 1.5175-1.5185
Dispersion (Abbe number): 39-50, preferably 42-46
Kinematic viscosity (mm²/s): 50-6000, preferably 800-1200
UV absorption (transmittance of 50% at d=10 mm): <400 nm, preferably <350 nm
Residual fluorescence (ex. 365 nm, em. 450 nm): equivalent <0.06 mg/l quinine sulfate in 1 N sulfuric acid, preferably <0.05 mg/l
Residual fluorescence (ex. 405 nm, em: 485 nm): equivalent <1.20 mg/l quinine sulfate in 1 N sulfuric acid, preferably <1.00 mg/l

The immersion liquid preferably meets the specifications of standard ISO 8036:2015-06.

An essential feature for excellent suitability of TCD acetals as main constituent of immersion liquids is the refractive index nₑ>1.5 and the simultaneously high Abbe number vₑ≥46, preferably vₑ≥47, more preferably vₑ≥50.

Also of significance is the good UV transparency of the TCD acetals, which is below 10% transmittance only at wavelengths below 320 nm for a layer thickness of 10 mm.

### EXAMPLES

### Example 1: Acetalization of Tricyclodecanedimethanol

There follows a description of the acetalization of tricyclodecanedimethanol (3(4),8(9)-bis(hydroxymethyl)tricyclo[5.2.1.0^{2,6}]decane; “TCD-DM” hereinafter) with 3,4-dihydro-2H-pyran (DHP), in each case at room temperature, for preparation of the corresponding bisacetal 3(4),8(9)-bis(tetrahydropyran-2-yloxymethyl)tricyclo[5.2.1.0^{2,6}]decane (“bisacetal P” hereinafter) with various acids as catalyst, which proceeds according to the following scheme:

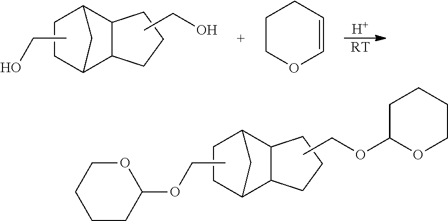

### Example 1a: Preparation of Bisacetal P with Concentrated Hydrochloric Acid

20 g of TCD-DM (0.1 mol; 1.00 eq.) and 28 ml of DHP (25.96 g; 0.31 mol; 3.08 eq.) in 300 ml of ethyl acetate formed an initial charge. Subsequently, 0.25 ml of conc. HCl (3 mmol; 0.03 eq.) was added. After 2 h, the reaction mixture was washed with 100 ml of 1M NaOH and twice with 100 ml of distilled water. The organic phase was dried over sodium sulfate and the solvent was removed under reduced pressure. A clear, colourless, oily liquid was obtained.

### Example 1b: Preparation of Bisacetal P with Concentrated Sulfuric Acid

20 g of TCD-DM (0.1 mol; 1.00 eq.) and 36.30 ml of DHP (33.65 g; 0.40 mol; 4.00 eq.) in 300 ml of ethyl acetate formed an initial charge. Subsequently, 0.21 ml of conc. H₂SO₄ (4 mmol; 0.04 eq.) was added. After 2 h, the reaction mixture was washed with 100 ml of 1M NaOH and twice with 400 ml of distilled water. The organic phase was dried over sodium sulfate and the solvent was removed under reduced pressure. A clear, pale yellowish, oily liquid was obtained.

### Example 1c: Preparation of Bisacetal P with Para-Toluenesulfonic Acid

300 g of TCD-DM (1.5 mol; 1.00 eq.) was dissolved in 500 ml of ethyl acetate. Subsequently, 2.55 g of para-toluenesulfonic acid (0.015 mol; 0.01 eq.) was added, and 306.76 ml of DHP (284.37 g; 3.38 mol; 2.25 eq.) was rapidly added dropwise. 2 h after commencement of the dropwise addition, the reaction was ended and the solution was transferred to a separating funnel. The reaction mixture was washed therein once with a solution of 2 g of NaOH in 150 ml of water and twice with about 300 ml of dist. water. The organic phase was dried over sodium sulfate and the solvent was removed under reduced pressure. The product was purified further by distillation.

### Example 2: Production of an Immersion Liquid

82.4 per cent by weight of the bisacetal P prepared by Example 1 was mixed with 17.6 per cent by weight of diisopropylnaphthalene and formulated to give the finished immersion liquid.

The immersion liquid thus obtained had the properties specified in the table below and was in accordance with standard ISO 8036:2015-06 (especially with regard to the Abbe number of 44±3).

| Refractive index nₑ (23° C.) | 1.5180 | |
|---|---|---|
| Dispersion/Abbe number vₑ (23° C.) | 44.13 | |
| Dynamic viscosity (25° C., 25 mm, 1°) | 1141.6 | mPa · s |
| Kinematic viscosity (calculated) | 1071.9 | mm²/s |
| Fluorescence (ex. 405 nm, em. 485 nm) | equivalent to 0.751 mg/l quinine | |
| | sulfate in 1N sulfuric acid | |
| Fluorescence (ex. 365 nm, em. 450 nm) | equivalent to 0.038 mg/l quinine | |
| | sulfate in 1N sulfuric acid | |
| UV-VIS transmittance of 50% at | at 341.19 | nm |
| d = 10 mm | | |
| Density (20° C.) | 1.065 | g/cm³ |

1. An immersion liquid, comprising:
one or more acetals of tricyclodecane alcohols as a main constituent,
wherein a tricyclodecane alcohol is a compound of which the structure has the base skeleton of tricyclo[5.2.1.0^{2,6}]decane and at least one OH group, and wherein all OH groups of the tricyclodecane alcohol have been acetalized in the acetal.

2. The immersion liquid according to claim 1, comprising one or more acetals of 8-hydroxymethyltricyclo[5.2.1.0^{2,6}]decane or 3(4),8(9)-bis(hydroxymethyl)-tricyclo[5.2.1.0^{2,6}]decane as the main constituent.

2. The immersion liquid according to claim 2, comprising 8-tetrahydropyran-2-yloxymethyltricyclo[5.2.1.0^{2,6}]decane and/or 3(4),8(9)-bis(tetrahydropyran-2-yloxymethyl)tricyclo[5.2.1.0^{2,6}]decane as the main constituent.

4. The immersion liquid according to claim 1, further comprising:
one or more high-boiling liquid(s) as secondary constituents.

5. The immersion liquid according to claim 4, wherein the one or more high-boiling liquids are selected from the group consisting of paraffin oil, plasticizers, polypropylene glycols, dioctyl sebacate, diisopropylnaphthalene, di(propylene glycol-1,2) benzoate, and mixtures thereof.

6. The immersion liquid according to claim 1, wherein a proportion of the one or more acetals of tricyclodecane alcohols is more than 50 percent by weight.

7. 3(4),8(9)-Bis(tetrahydropyran-2-yloxymethyl)tricyclo[5.2.1.0^{2,6}]decane or 8-tetrahydropyran-2-yloxymethyltricyclo[5.2.1.0^{2,6}]decane.

8. A method, comprising:
preparing an immersion liquid with one or more acetals of tricyclodecane alcohols as a main constituent.

9. A process for preparing an acetal of a tricyclodecane alcohol, comprising:
reacting a tricyclodecane alcohol with an acetalizing agent in the presence of an acid.

10. The process according to claim 9, wherein the reaction is effected at a temperature within a range from 20 to 30° C.

11. The process according to claim 9, wherein the acetalizing agent is 3,4-dihydro-2H-pyran.

12. The immersion liquid according to claim 1, wherein the immersion liquid is an immersion liquid for microscopy.

13. The immersion liquid according to claim 6, wherein the proportion of the one or more acetals of tricyclodecane alcohols is at least 60 percent by weight.
